(19) United States
(12) Patent Application Publication
(10) Pub. No.: US 2012/0039858 A1
(43) Pub. Date: Feb. 16, 2012

(54) METHODS AND COMPOSITIONS FOR INHIBITING PROPAGATION OF VIRUSES USING RECOMBINANT TETHERIN CONSTRUCTS

(75) Inventor: Paula Cannon, Los Angeles, CA (US)

(73) Assignee: CHILDRENS HOSPITAL LOS ANGELES, Los Angeles, CA (US)

(21) Appl. No.: 13/124,559

(22) PCT Filed: Oct. 16, 2009
(86) PCT No.: PCT/US2009/061025

§ 371 Date: Apr. 15, 2011

Related U.S. Application Data
(60) Provisional application No. 61/196,291, filed on Oct. 16, 2008.

(51) Int. Cl. A61K 48/00; C07H 21/00; A61P 31/12; C12N 1/00; C12N 5/10; A61P 31/18; C07K 19/00; C12N 15/12

(52) U.S. Cl. 424/93.21; 530/350; 536/23.4; 435/243; 435/325; 435/369

(57) ABSTRACT

The present invention provides chimeric protein constructs having anti-viral activity, compositions and methods of using them, and nucleic acids encoding them. The chimeric proteins include an extracellular domain of a Tetherin protein fused to the transmembrane domain, and optionally cytoplasmic tail, of a different protein. The chimeric proteins have normal anti-viral tethering activity but are resistant to inhibition by anti-Tetherins. Ex vivo methods of gene therapy are also provided.

### CROSS-REFERENCE TO RELATED APPLICATIONS

The present application relies on and claims the benefit of the filing date of U.S. provisional patent application No. 61/196,291, filed 16 Oct. 2008, the entire disclosure of which is hereby incorporated herein by reference.

### STATEMENT OF GOVERNMENT INTEREST

This invention was made partially with U.S. Government support from the United States National Institutes of Health under Grant Number 5R01 AI068546. The U.S. Government has certain rights in the invention.

### BACKGROUND OF THE INVENTION

1. Field of the Invention

The present invention relates to the fields of medicine and in particular gene therapy. More specifically, the invention relates to recombinant chimeric proteins, compositions, and methods for treatment of viral infections, such as infections by enveloped viruses. Exemplary viruses include human immunodeficiency virus (HIV).

2. Description of Related Art

Certain human cells posses an activity that inhibits the release of retroviruses and other enveloped viruses from those cells. The activity is linked to molecules that tether the viral particles to the cells, and those molecules have therefore been termed “Tetherins”. The human protein BST-2/CD317/HM1.24/Tetherin has recently been identified as a cellular factor that tethers newly budded HIV particles at the surface of a cell, and thereby reduces the yield of infectious virions (Neil et al., 2008, Nature 451:425-430; Van Damme et al., 2008, Cell Host. Microbe 3:245-252). Its molecular mechanism of action is presently unknown, but the fact that Tetherin exists as a homodimer, with each monomer anchored in the plasma membrane through both a membrane-spanning sequence and a GPI anchor, has led to the suggestion that the protein physically links viral and cellular membranes, preventing viral particle release from infected cells. Tetherin also restricts the release of enveloped viruses other than HIV, including other lentiviruses, retroviruses, filoviruses, herpesviruses, and arenaviruses, suggesting that it may be part of an innate cellular defense against enveloped viruses.

HIV codes for two distinct proteins that counteract the action of Tetherin, the HIV-1 Vpu protein and the HIV-2 Env protein (Anti-Tetherins) (Strebel et al., 1988, Science 241:1221-1223; Bour et al., 1996, J. Virol. 70:8285-8300; Noble et al., 2005, J. Virol. 79:3627-38). In addition, the Kaposi's sarcoma-associated herpesvirus (KSHV), which can be a significant cause of pathology in HIV-infected individuals, also targets Tetherin through the action of its K5 protein. There thus appears to be an evolutionarily developed response by viruses to overcome the Tetherin-directed cellular response to viral infection.

Very few drugs or viral inhibitors are known that act at late stages of the HIV life-cycle, such as at virus release. In part, this reflects the fact that these stages are difficult targets to analyze in standard high throughput screens (HTS). Typically such studies have used the secretion of virus-like particles (VLPs) into cell culture supernatants as the assay endpoint, to be measured after concentration and quantitation using enzyme assays (e.g., reverse transcriptase activity), by measurement of HIV antigens, or through the inclusion of covalently linked enzymatic reporters in the VLPs (e.g., alkaline phosphatase or β-lactamase). These assays are somewhat cumbersome, requiring harvesting and concentration of supernatants, and this significantly limits their application to HTS formats.

Diseases and disorders affecting humans and other mammals traditionally have been treated using small molecules (i.e., drugs). Recently, biologics (i.e., protein-based substances) have been used in place or in addition to drugs. As an alternative to traditional “drug” therapies and “biologics” therapies for treatment of diseases and disorders, gene therapy techniques have been developed. Typically, gene therapy treatments have been used to treat diseases and disorders having a genetic basis. For example, diseases and disorders resulting from the absence of a functional protein have been treated by supplying a functional gene to the subject, which is expressed in target cells and supplies the required functional protein. However, to date, the use of gene therapy to treat viral infections has not been established.

### SUMMARY OF THE INVENTION

In view of the tremendous medical, economic, and societal impact of viral infections, including HIV infections, in humans, new methods of treatment are needed. The present invention provides chimeric (also referred to herein as “fusion”, “recombinant”, or “engineered”) proteins for treatment of viral infections, and in particular infections caused by enveloped viruses. In general, the chimeric proteins of the invention include an extracellular domain (also referred to herein as an “ectodomain”) of a Tetherin protein (typically including a GPI anchor) fused to a functional membrane targeting and anchoring domain of another protein, or a mutated membrane targeting and anchoring domain of the same Tetherin protein from which the extracellular domain derives. In certain exemplary embodiments, the membrane targeting and anchoring domain comprises a transmembrane domain of another protein. In other exemplary embodiments, the membrane targeting and anchoring domain comprises the transmembrane domain and at least part of the cytoplasmic domain of another protein. In yet other exemplary embodiments, the membrane targeting and anchoring domain comprises the transmembrane domain, at least part of the cytoplasmic domain, and one to ten residues of the ectodomain of another protein. The chimeric proteins retain the anti-viral activity of the Tetherin extracellular domain, are properly inserted and retained in a cellular membrane, and are resistant to viral inactivation by anti-Tetherin proteins produced by viruses during the infection/propagation cycle. The invention identifies the transmembrane (TM) domain of Tetherin as an important site for inhibition by anti-Tetherin molecules produced by viruses, and the combination of the TM domain and at least part of the cytoplasmic domain as a highly advantageous combination site for inhibition. The chimeric proteins of the invention have altered TM domains, cytoplasmic (C) domains, or TM and C (TMC) domains, which render the chimeric proteins resistant to inhibition by anti-Tetherins.

The chimeric proteins of the invention can be used to block budding of enveloped viruses from infected cells. They thus have anti-viral activity and can be used in methods of treatment of viral infections. In general, the methods of treatment of viral infections include providing a chimeric protein to a cell that is infected or susceptible to infection by a virus under conditions where the chimeric protein localizes to the cell membrane of the cell. While the step of providing the chimeric protein to the cell can be any action that results in localization of the protein on the cell membrane, in exemplary embodiments of the invention, the step of providing includes introducing into the cell a nucleic acid encoding the chimeric protein, and allowing the cell to express the chimeric protein.

The chimeric proteins of the invention can be expressed from recombinant nucleic acids, can be produced chemically, or can be produced partially by each method and combined to form a functional protein. In general, recombinant nucleic acids according to the invention include the coding sequence for a Tetherin extracellular domain or a portion thereof having anti-viral tetherin activity. The extracellular domain or portion thereof is fused to a TM, C, or TMC that does not have the cognate sequence for inhibition through the activity of a viral anti-Tetherin molecule. Typically, the TM, C, or TMC domain is selected from another protein known not to have a target sequence for inhibition by an anti-Tetherin of interest. However, the TM, C, or TMC, in embodiments, can be fully artificial or can be a mutated form of a naturally occurring TM, C, or TMC of a protein of interest (e.g., a mutated form of the Tetherin from which the extracellular domain derives) or can be sequences from non-human versions of Tetherin that are not susceptible to human viral anti-Tetherin factors. In embodiments, the recombinant nucleic acids can include an N-terminal, cytoplasmic tail fused to the TM domain encoding sequence. The nucleic acids of the invention can include additional functional elements, including, but not limited to, promoters or other elements for control of expression of the fusion coding region. The nucleic acids can thus take the form of viral genomes, plasmids, phagemids, or other vectors for delivery, maintenance, and/or expression of exogenous nucleic acids in a cell.

### BRIEF DESCRIPTION OF THE DRAWINGS

The accompanying drawings, which are incorporated in and constitute a part of this specification, illustrate embodiments of the invention and provide experimental support for embodiments of the invention, and together with the written description, serve to explain certain principles of the invention.

FIG. 1 shows an amino acid sequence alignment of selected Tetherins from primate species. The approximate transmembrane regions of the proteins are indicated by asterisks.

FIG. 2, Panel A, depicts a cartoon representation of a wild-type Tetherin (protein on the left) and a chimeric protein according to an embodiment of the invention (protein on the right). In the chimeric protein, the Tetherin-derived extracellular domain is represented by a solid line (the GPI anchor represented by a sphere at the C-terminus), the TM region of the human TfR1 is represented by the membrane-spanning ovoid shape, and the cytoplasmic tail and a short extracellular portion of the human TfR1 is represented by a dotted line.

FIG. 2, Panel B, depicts the amino acid sequence of the chimeric protein depicted in FIG. 2A, in which the human TfR1-derived sequence is presented in italics and the Tetherin-derived sequence is underlined. The approximate TM domain is depicted in bold typeface.

FIG. 2, Panel C, depicts the amino acid sequence of another chimeric protein according to the invention, in which the macaque Tetherin TMC sequence is fused to the human Tetherin extracellular domain. In the figure, the macaque TMC is presented in italics and the Tetherin-derived sequence is underlined. The approximate TM domain is depicted in bold typeface.

FIG. 3, shows Western blot analysis of cell lysates and virus-like particle (VLP) pellets using anti-HIV-1-p24 antibodies. The left panel shows that expression of Tetherin VLP decreases VLP release, and that this effect is counteracted by Vpu. The center panel shows that a construct of the invention (“TT”) restricts VLP release, but is not counteracted by Vpu. The right panel shows that a different construct of the invention (“MT”) restricts VLP release, but is not counteracted by Vpu. The TT and MT constructs are shown not to be inhibited by HIV Vpu protein.

### DETAILED DESCRIPTION OF VARIOUS EMBODIMENTS OF THE INVENTION

Reference will now be made in detail to various exemplary embodiments of the invention. It is to be understood that the following detailed description of embodiments is provided to give the reader a better understanding of certain embodiments and features of the invention, and is not to be considered as a limitation on the scope or content of the invention, as broadly disclosed herein.

The current method of choice for treatment of chronic viral infections, including HIV infections, involves administering to an infected subject one or more small molecule compounds (i.e., drugs) that interrupt or otherwise diminish viral replication within cells of the patient. However, such treatments are costly and short-lived, requiring repeated doses to maintain viral titers at an acceptable level. Alternatives to common anti-viral treatment regimens have been investigated, and success has been achieved through gene therapy techniques. Targeting of blood cells for gene therapy has been shown to be a viable option for treatment of not only HIV, but neoplasias as well. (See, for example, Auiti, A. et al., N. Engl. J. Med., 360(5):447-458, Jan. 29, 2009; Varela-Rohena, A. et al., Immunol. Res., 42(1-3):16-181, 2008; Garcia, J. M. et al., Allergol. Immunopathol. (Madr), 35(5):184-192, September-October 2007; and Burke, B. et al., Journal of Leukocyte Biology, 72:417-428, 2002.) However, targets for gene therapy to treat viral infections, including HIV infections, are limited and often are virus-specific. The present invention provides a novel target for treatment of viral infections, which is suitable as a target for infections of a variety of viruses, and in particular enveloped viruses.

It is known in the art that certain human cells (i.e., HeLa cells) can restrict the release of HIV-1 virus-like particles (VLPs), while simian (Cos-7) cells do not. The basis for this restriction has recently been identified as the human BST-2/CD317/1-IM1.24/Tetherin (“Tetherin”). Both HIV-1 Vpu and the HIV-2 Env can counteract this restriction and thereby increase the level of VLPs released from HeLa cells. Likewise, the KSHV K5 protein shares this activity. It has also been found that adding human Tetherin to simian Cos-7 cells profoundly restricts VLP release, and that this restriction can be counteracted by both Vpu and HIV-2 Env (known generally as “anti-Tetherins”). It thus is apparent that interplay between Tetherin and anti-Tetherin molecules has an important role in the viral replication and infection cycle. During investigations to better understand the portions of Tetherin that are involved in inhibition of Tetherin activity by anti-Tetherins, the present inventor has determined that the TM domain of Tetherin is involved in, if not responsible for, the inhibitory activity of certain anti-Tetherins (e.g., HIV-1 Vpu, HIV-2 Env), that the cytoplasmic domain is targeted by others (e.g., KSHV K5), and the ectodomain is involved for others (e.g., Ebola GP, HIV-2 Env). The inventor has also determined that an anti-Tetherin produced by a virus can specifically inhibit the cell localization activity of the Tetherin of the virus host species. Identification of these regions of Tetherin, and the specificity of a particular viral anti-Tetherin for particular domains of a Tetherin of the host species for the virus, forms a basis for the present invention.

The anti-viral activity of a Tetherin results from proper cellular localization of the Tetherin at the cell surface, and subsequent inhibition of release of viral particles. Tetherin cell-surface localization is primarily dictated by the TM domain, and can be assisted by ancillary anchoring by a C-terminal GP1 anchor, while anti-budding activity is primarily dictated by the extracellular domain, which is often referred to as the ectodomain. The anti-viral budding activity of Tetherin is reduced by anti-Tetherin proteins encoded by viruses, such as by the Vpu protein of HIV-1 and the K5 protein of KSHV. According to the present invention, a chimeric protein is provided that has anti-viral budding activity by way of a Tetherin ectodomain or an active portion thereof but is resistant to a selected anti-Tetherin by way of a TM domain that is incompetent for inhibition via the anti-Tetherin.

In a first aspect of the invention, chimeric proteins are provided. The chimeric proteins include (1) an extracellular domain derived from a Tetherin that is capable of binding to a target virus and (2) a TM, C, or TMC domain that is capable of directing and maintaining the chimeric protein at a cell surface. The cell-surface localizing activity of the TM domain or the TMC combined domains is not completely inhibited as a result of expression of an anti-Tetherin expressed by the target virus. In essence, the specificity of a particular anti-Tetherin for the TM, C, or TMC domains of a particular Tetherin is used to develop chimeric proteins having Tetherin activity but not being substantially inhibited by an anti-Tetherin.

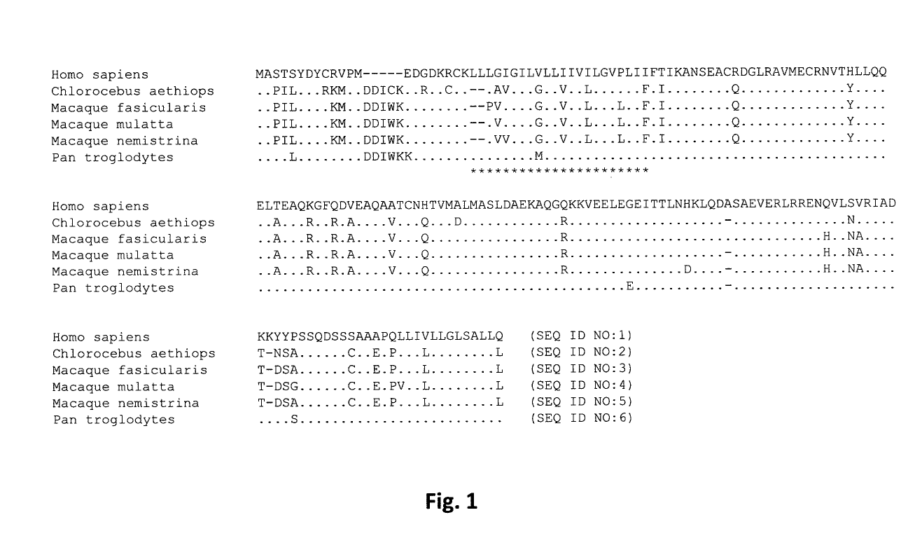

Although the primary amino acid sequences of Tetherins among different species vary, the predicted secondary and tertiary structures of Tetherins are highly conserved among species. A comparison of Tetherin sequences from various primate species is presented in FIG. 1. As can be seen from the figure, Tetherins generally contain an N-terminal cytoplasmic domain or tail, a TM domain (noted by asterisks in the figure), and an extracellular domain that contains a GPI anchor insertion sequence. Using the primary amino acid sequence as a guide, one may select any extracellular domain sequence having viral binding and retention activity for use in a chimeric protein according to the invention. It is to be understood that, while use of a wild-type or naturally-occurring sequence of a Tetherin is encompassed by the present invention, the invention is not limited to use of any specific sequence. Rather, one may select or engineer any particular extracellular sequence desired, as long as the selected or engineered sequence possesses anti-viral activity.

Thus, for example, the chimeric protein may include the extracellular domain of human Tetherin presented in FIG. 1. In some embodiments, the chimeric protein includes residues 44-180 of SEQ ID NO:1. In some embodiments, the extracellular domain of the chimeric protein shows some, but not exact, primary sequence identity to the human Tetherin sequence presented in FIG. 1. For example, one or more point mutations may be introduced into the naturally-occurring human Tetherin sequence, or one or more deletions or insertions may be introduced. Any variation from the naturally-occurring sequence may be introduced, the limitation being that the resulting extracellular domain must have a detectable level of target virus anti-budding activity. Generally, point mutations will result in conservative amino acid substitutions according to well-established principles of protein biochemistry. Further, as with point mutations, insertions and deletions are limited only by their functional effect on the anti-viral budding activity of the chimeric protein. Insertions and deletions are thus not limited in length. However, in some embodiments, insertions or deletions will be limited in size, for example to insertion or deletion of 1-15 residues. It is to be understood that fusion of a TM or TMC to the N-terminus of the extracellular domain is not an “insertion” according to the present invention.

A chimeric protein according to the invention thus may have, for example, an extracellular domain showing about 50% or greater primary sequence identity to residues 44-180 of SEQ ID NO:1, such as about 60% or greater, about 70% or greater, about 80% or greater, about 85% or greater, about 90% or greater, about 95% or greater, or about 99% or greater. Of course, any particular value within these ranges is contemplated by the invention, and those of skill in the art will immediately recognize each particular value without the need for each to be recited herein. Percent identity can be determined by alignment of the sequence of SEQ ID NO:1 with the derived sequence, maximizing identity of residues along the two sequences, and determining the percent identity with reference to the sequence of residues 44-180 of SEQ ID NO:1. Various techniques for introducing mutations into a protein are known and widely practiced in the art of molecular biology. Any suitable technique may be used to create the sequences of the chimeric protein of the invention, and the practitioner may select a desired technique based on any number of parameters. Furthermore, those of skill in the art can easily select for engineered sequences having the desired anti-viral budding activity using techniques known in the art and/or disclosed herein. That is, assays for Tetherin anti-viral budding activity are known in the art, and screening for engineered sequences having a desired activity can be performed using routine and straightforward techniques.

Although engineering of extracellular domain sequences has been discussed with reference to the human Tetherin sequence above, it is to be understood that the same concepts apply to Tetherins from all species. For example, a chimeric protein based on the chimpanzee Tetherin sequence can be created using the principles discussed above. Likewise, a chimeric protein based on a macaque Tetherin can be created. The chimeric proteins of the invention are thus not limited to human sequences or the sequences specifically presented herein, but rather are broadly directed to all proteins based on Tetherin sequences. In preferred embodiments, the chimeric protein includes a Tetherin extracellular domain from human Tetherin, or a portion of that domain that is sufficient for binding and retaining budding viruses at the cell surface. In a preferred embodiment, the Tetherin extracellular domain or active portion thereof is capable of binding and retaining budding HIV virus. In another preferred embodiment, the Tetherin extracellular domain or active portion thereof is capable of binding and retaining KSHV.

In addition to the extracellular domain, the chimeric proteins of the invention include a TM domain or a TMC combination. The TM or TMC of a chimeric protein according to the invention is fused at its C-terminus to the N-terminus of the extracellular domain (either directly or by way of a linker sequence). While any technique for fusing the two sequences is contemplated by the invention, in preferred embodiments, the two domains are fused by way of fusion of their respective coding sequences in-frame in a nucleic acid construct. In general, the TM or TMC is any amino acid sequence that functions to localize a protein at the cell surface by way of embedding of the TM or TMC within and across a cell surface membrane. The only general restriction on the sequence of the TM or TMC is that it must not be completely inhibited in its membrane-localizing activity as a result of the activity of a viral anti-Tetherin that is expressed by a virus against which the extracellular domain of the chimeric protein has activity. For example, in embodiments where the extracellular domain of the chimeric protein specifically inhibits budding of HIV from human cells, cell surface localization of the chimeric protein via the TM or TMC cannot be completely inhibited by an HIV anti-Tetherin, such as Vpu.

In exemplary embodiments, the TM or TMC of the chimeric protein is a TM domain or a combination of TM and C domains derived from a protein other than the Tetherin from which the extracellular domain is derived. Thus, for example, where the extracellular domain is derived from human Tetherin, the TM and/or TMC domains are not derived from human Tetherin. However, in some embodiments, the TM or TMC is derived from the same Tetherin as the extracellular domain, but has been mutated such that it is not completely inhibited in its cell-surface localization activity by a viral anti-Tetherin expressed by the virus against which the extracellular domain has activity. For example, where the extracellular domain is derived from residues 44-180 of SEQ ID NO:1, the TM domain may be derived from about residue 22 to about residue 43 of SEQ ID NO:1, but include one or more point mutations, insertions, or deletions that render it at least partially resistant to the inhibitory activity of a selected anti-Tetherin, such as HIV-1 Vpu. Likewise, where the extracellular domain is derived from residues 44-180 of SEQ ID NO:1, the TMC domain may be derived from about residue 1 to about residue 43 of SEQ ID NO:1, but include one or more point mutations, insertions, or deletions that render it at least partially resistant to the inhibitory activity of a selected anti-Tetherin, such as KSHV protein K5. In exemplary embodiments, the TMC includes residues of SEQ NO:1 from about residue 3 to about residue 43, from about residue 10 to about residue 43, and from about residue 15 to about residue 43. As with the extracellular domain, the TM domain may be derived from any Tetherin TM domain, for example, a TM domain disclosed in FIG. 1. The TM domain also may be derived from a TM domain not exemplified in FIG. 1, using the comparison of FIG. 1 to guide the selection of residues to be included in the TM domain (commercially available computer programs may also be used to develop an appropriate TM domain of a Tetherin). In some embodiments, the TM domain is a TM domain derived from a Tetherin from a species that is different than the species from which the extracellular domain is derived. In a non-limiting example discussed in detail below, the TMC has the sequence of residues 1-46 of Macaque mulatta (i.e., residues 1-46 of SEQ ID NO:4) Tetherin, which is fused to the extracellular domain of human Tetherin.

In general, the TM domain will be about 18-24 residues in length and contain residues known to be appropriate for a TM domain. Those of skill in the art are fully aware that transmembrane domains (also referred to in the art as membrane-spanning regions) share certain physical characteristics. For example, they typically are defined by lengths of about 20 residues, are generally comprised of hydrophobic or non-polar residues, and generally do not include residues that cause inflexible bends or turns (e.g., typically do not comprise proline). Substitutions that may be made include, but are not limited to substitutions of one or more of the following amino acids with others of the group: alanine, cysteine, glycine, isoleucine, leucine, methionine, phenylalanine, proline, tryptophan, and valine. While not limited to any particular substitutions/mutations, examples of residues that may be varied to provide resistance to certain anti-Tetherins include residues of the human Tetherin (and corresponding residues from Tetherins of other species) between residue 22 and 43 of SEQ ID NO:1 other than: G25, 126, 128, L29, V30, 133, 134, 136, P40, and 143.

The TM or TMC of the chimeric protein is preferably derived from a protein other than a Tetherin. For example, it can be derived from any of a number of cell-surface proteins known in the art, including, but not limited to cell surface receptors. Non-limiting examples include TM and TMC from Type land Type II membrane proteins. Specific, non-limiting examples of TM and TMC are those derived from a Type I protein, such as that of CD4 or CD8, and those derived from a Type II protein, such as that of the Transferrin Receptor Type I protein (TfR1). TM and TMC sequences from proteins other than Tetherins are preferred because the likelihood of inhibition of cell-surface localization as a result of anti-Tetherin expression by a virus is dramatically reduced or completely avoided.

In describing the chimeric protein of the invention, reference has been made to inhibition of cell-surface localization of the chimeric protein by an anti-Tetherin. As used herein, the term inhibition is used to describe the amount of chimeric protein found on the cell surface when co-expressed with a relevant anti-Tetherin, relative to the amount found when the naturally-occurring TM or TMC is expressed in combination with the extracellular domain normally associated with the TM or TMC. Thus, the term “complete inhibition” is not to be interpreted as requiring that no Tetherin sequences can be found at the cell surface. Rather, it is to be interpreted as meaning the amount of Tetherin sequences of the chimeric protein found at the cell surface is insignificantly different than the amount seen when a naturally-occurring Tetherin (comprising the corresponding naturally-occurring TM or TMC and extracellular domains) is co-expressed with the anti-Tetherin. In addition, where used herein, the term “complete resistance” is used, it is meant that the amount of chimeric protein found at the cell surface, when co-expressed with an anti-Tetherin, is insignificantly different than the amount of a naturally-occurring Tetherin from which the chimeric protein is derived found on the cell surface in the absence of the anti-Tetherin.

The chimeric proteins of the invention preferably are completely resistant to the anti-Tetherin expressed by the target virus. However, because the amount of resistance will vary depending on the particular target virus, the particular Tetherin, the particular TM domain, the type of cell infected by the virus, and the general environment of the cell, the invention contemplates that, in embodiments, resistance of the chimeric protein to the anti-Tetherin will not be complete. The chimeric protein of the invention thus may be characterized as less inhibited by the selected anti-Tetherin than a naturally occurring (e.g., wild-type) Tetherin from which its ectodomain is derived. Inhibition of the chimeric protein by the anti-Tetherin (as compared to inhibition of a naturally-occurring Tetherin from which it is derived) may be any amount detectable, such as, for example, less than 1% inhibition, less than 2% inhibition, less than 5% inhibition, less than 10% inhibition, less than 20% inhibition, and less than 50% inhibition. Stated another way, the chimeric proteins of the invention are less inhibited by a selected anti-Tetherin than is a naturally-occurring Tetherin from which its sequence is derived. For example, the chimeric protein may be at least 10% less inhibited, at least 20% less inhibited, at least 50% less inhibited, at least 70% less inhibited, or about 100% less inhibited.

The term inhibition is also used herein to describe the activity of the chimeric proteins on viral budding and release from an infected cell. This type of inhibition is separate and distinct from inhibition relating to anti-Tetherin activity on the chimeric protein, although the two types of inhibition are related. More specifically, inhibition of cell-surface localization by an anti-Tetherin is related, but not necessarily equated, with inhibition of virus budding and release. On the one hand, a chimeric protein that is inhibited by an anti-Tetherin to some degree will also have reduced inhibitory activity against viral release by its absence on the cell surface. However, the inhibitory ability of a chimeric protein may also be reduced due to mutations in the extracellular domain of the chimeric protein, which can reduce its ability to bind and retain budding virus on the cell surface. While chimeric proteins having full viral retention activity (as compared to a naturally-occurring Tetherin from which its extracellular domain is derived) are preferred, the invention encompasses chimeric proteins with reduced viral release inhibition. Viral inhibition by the chimeric protein may be at least 10%, at least 20%, at least 50%, at least 70%, or 100% (i.e., indistinguishable from the naturally-occurring Tetherin).

The chimeric protein of the invention can, but does not necessarily, include additional amino acid residues N-terminal to the TM domain. In the wild-type Tetherin, an N-terminal cytoplasmic domain or “tail” is present. In the human Tetherin, the N-terminal tail is represented by residue 1 through about residue 21 (see FIG. 1, for example). This N-terminal tail is generally conserved among Tetherins from various species, and it is postulated that it might play a role in cell-surface localization of the Tetherin. According to the present invention, the N-terminal cytoplasmic domain of a Tetherin may be deleted, retained, or replaced. In preferred embodiments, the N-terminal cytoplasmic domain is deleted or replaced. In less preferred embodiments, the N-terminal cytoplasmic domain is retained, but is preferably mutated at one or more residues. In exemplary embodiments, the N-terminal cytoplasmic domain is replaced by a soluble domain from another protein, such as a cytoplasmic domain from a different membrane protein. As with the TM domain, numerous cytoplasmic domains are known in the art, and the practitioner is free to choose any suitable cytoplasmic domain desired.

The cytoplasmic domain, if present, is fused at its C-terminus to the N-terminus of the TM domain, either directly or via a linker. Any method of fusing is encompassed by the invention, with fusion by way of in-frame fusion of corresponding coding regions of nucleic acids being preferred. The length of the cytoplasmic domain is not particularly limited.

As is evident from the disclosure above, the chimeric proteins of the invention may include additional amino acid residues at the N-terminus or C-terminus. The chimeric proteins thus may consist of a particular amino acid sequence or comprise that sequence. The only limitation on the additional residues is that they not substantially interfere with the anti-viral release activity and the anti-Tetherin resistance activities of the chimeric proteins. The chimeric proteins thus may include one or more labels, which can be used for in vitro determination of cellular localization of the chimeric proteins. Numerous labels that are suitable for detecting proteins are known in the art, and the practitioner is free to select an appropriate label for a particular application. Non-limiting examples of labels include, but are not limited to, protein sequences having intrinsic detectable activity (e.g., fluorescent proteins), peptide antigens for detection with antibodies, enzymes that can participate in production of a detectable signal, and fluorescent tags.

The chimeric proteins of the invention can be expressed in cells, can be purified or isolated substances, or can be part of compositions. Where the proteins are part of compositions, the compositions are not particularly limited. They thus can be any of a number of liquid or solid compositions, comprising any other substances or combination of substances. In general, it is preferred that the substances present in the composition in addition to the chimeric proteins are compatible with the stability and activity of the chimeric proteins. Non-limiting examples of additional substances include solvents, such as water, glycerol, or organic solvents (e.g., methanol), buffers (e.g., Tris, MOPS, HEPES), and salts (e.g., sodium salts, potassium salts, magnesium salts). Additional non-limiting substances that can be present in compositions according to the invention include some or all of the substances necessary for detecting the presence of the chimeric proteins. Non-limiting examples include antibodies, enzymatic substrates, energy (e.g., electron or electromagnetic radiation) donors for fluorescence, and energy acceptors/re-emitters. In some embodiments, the compositions comprise cells or cell lysates. Yet again, in some embodiments, the compositions comprise protein purification fractions. In preferred embodiments for in vivo use, the chimeric proteins are formulated in compositions for delivery to a subject, such as a human patient suffering from a viral infection. In general, such compositions comprise the chimeric protein in an aqueous composition that includes one or more additional substances typically included in pharmaceutical or therapeutic compositions. Those of skill in the medical arts can easily devise appropriate pharmaceutical compositions based on standard, well established pharmacological parameters without the need for the various suitable substances to be specifically disclosed herein.

The chimeric proteins of the invention can be produced by way of total or partial chemical synthesis, but are preferably produced from recombinant nucleic acids. As such, one aspect of the invention is nucleic acids encoding the chimeric proteins. Nucleic acids include both double-stranded and single-stranded molecules, including double-stranded or single-stranded DNA and double-stranded or single-stranded RNA. The nucleic acid may be a hybrid of RNA and DNA. The nucleic acid thus may be mRNA or a nucleic acid derived therefrom, such as cDNA. According to the invention, the nucleic acids include a polynucleotide sequence encoding a TM or TMC fused in-frame to a polynucleotide sequence encoding a Tetherin extracellular domain, as detailed above. Standard, widely practiced methods of making fusion nucleic acids can be used to create the nucleic acids of the invention. Likewise, standard mutagenesis techniques can be used on nucleic acids to create chimeric proteins having desired amino acid sequences, as detailed above.

In embodiments, the nucleic acid of the invention consists of the coding sequence of a chimeric protein of the invention. In embodiments, the nucleic acid of the invention comprises the coding sequence of a chimeric protein, wherein the sequence includes the coding region of the protein and additionally includes one or more nucleotides at either or both ends of the coding sequence. In preferred embodiments, the nucleic acid comprises some or all of the regulatory elements required for expression of the chimeric protein in a chosen host cell. It thus may comprise promoters, transcription factor binding sites, and the like. For example, for expression in T cells, a T cell-specific promoter may be used. Use of a cell-specific promoter allows for improved control of expression of the chimeric proteins, and reduces potential side-effects of expression of the chimeric proteins in non-target cells. Another example is to use the HIV-1 LTR promoter which then limits expression of the anti-Tetherin to cells that have been infected by HIV-1 and are making the HIV-1 Tat protein which activates the HIV-1 LTR promoter. For example, for treatment of HIV infection in vivo, one may select to express a chimeric protein only in T cells or only in white blood cells. Alternatively, for treatment of herpesviruses in vivo, one may select to express a chimeric protein only in neural cells. Any number and combination of expression control elements may be included in the nucleic acids, and those of skill in the art are free to select appropriate and/or desired elements based on the particular intended use of the chimeric protein.

In embodiments, the nucleic acid is a vector for introduction and/or maintenance of the nucleic acid in a host cell. For example, the nucleic acid may be a plasmid suitable for insertion into a host cell and production of a chimeric protein. Likewise, the nucleic acid may be a viral genome, or portion thereof. Numerous vector backbones are known and commercially available, and any suitable vector backbone may be used in accordance with the present invention. Preferably, the vector is capable of being maintained in a host cell at least long enough to express the chimeric protein. In some embodiments, at least the coding region, more preferably the coding region plus expression control sequence(s), are stably inserted into the genome of a host cell. Thus, in embodiments, the nucleic acid is an engineered genome of a host cell. Where intended for insertion into a genome of a host cell, the nucleic acid can comprise one or more sequences for insertion into the host cell genome. For example, the nucleic acid can comprise insertion element sequences, viral insertion sequences, or sequences designed for homologous recombination at a specific site in a host genome.

As with other embodiments of the invention, because the nucleic acids of the invention encode non-naturally occurring proteins, the nucleic acids are likewise non-naturally occurring. In certain embodiments, the nucleic acids are purified or isolated from other substances, such as cellular molecules.

The nucleic acids of the invention include coding sequences for the chimeric proteins of the invention. Exemplary amino acid sequences for the Tetherin extracellular domain (and a Tetherin TM or TMC, if used) of the chimeric proteins are provided herein and/or can be found in the literature. For example, the nucleic acid sequences for the Tetherin sequences can be taken from GenBank Accession Numbers: NM_{—}004335, FJ943431, FJ345303, FJ868941, CJ479048, DY743778, and XP_{—}512491. Alternatively, the nucleic acid sequence can be a nucleic acid sequence according to SEQ ID NO:7, which provides a nucleic acid sequence encoding the sequence of SEQ ID NO:8, which is a specific chimeric protein according to the invention (discussed in detail below). Yet again, the nucleic acid sequence can be one that encodes the chimeric protein of SEQ ID NO:9. Of course, due to the degeneracy of the genetic code, alterations in the precise sequences discussed herein can be made without altering the encoded amino acid sequences. In general, the coding sequences of the nucleic acids of the invention can easily be determined using widely available computer programs based on the selected amino acid sequences of the chimeric proteins and the genetic code.

It is common in the art to describe nucleic acids with regard to sequence identity. In the present situation, it is to be noted that the invention contemplates nucleic acids that have the functionality described herein and also have a particular level of sequence identity to specifically disclosed sequences. While the invention is not limited in any way by or to the specifically disclosed sequences, in embodiments the nucleic acids can be described as those showing 50% or more sequence identity with a specifically disclosed sequence, as calculated over the length of the disclosed sequence. In embodiments, the level of sequence identity is about 75% or more, about 90% or more, about 95% or more, about 97% or more, or about 99% or more. Those of skill in the art are to understand that each particular value falling within 50% to 100% (e.g., 51%, 52%, 53%, etc.) is specifically envisioned as a value according to the invention, and the need to recite each particular value is not necessary to capture this subject matter. Those of skill in the art can derive suitable nucleic acid sequences that encode chimeric proteins of the invention based on the genetic code with ease. For example, publicly available computer programs can be used to reverse translate the polyamino acids provided herein to arrive at exemplary nucleic acids according to the invention. Likewise, those of skill in the art can make suitable nucleic acids using standard molecular biology techniques. Because those of skill in the art are fully capable of producing all of the nucleic acids encompassed by the present invention, each particular sequence need not be disclosed herein.

The invention also provides biological cells. In general, the cells include a chimeric protein or recombinant nucleic acid of the invention. In some embodiments, the cells include both. Cells according to the invention can be any type of cell, including prokaryotic and eukaryotic cells. Cells according to the present invention comprise non-naturally occurring nucleic acids, proteins, or both. They are thus not products of nature. Likewise, in embodiments, the cells are isolated or purified away from some or all other cells in their natural environment (e.g., blood cells removed from a body for ex vivo manipulation). While not limited to any particular or single use, typically, prokaryotic cells according to the invention are used for production of nucleic acids according to the invention (e.g., plasmids, phagemids). Cells containing a nucleic acid of the invention are broadly referred to herein as recombinant cells or host cells. Cells for production and/or assay of the chimeric proteins of the invention are typically eukaryotic cells, provided in vitro (e.g., tissue culture cells), in vivo (e.g., in the body of a patient), or ex vivo (i.e., cells removed from a subject for treatment outside of the body and return to the body). Among the many uses for the cells of the invention, mention can be made of protein or nucleic acid production, research, and therapeutic treatment of viral infections.

Where used in vitro, the cells of the invention can be used for research purposes, for example in generating chimeric proteins and screening them for activity. For example, a chimeric protein can be engineered and then tested in vitro in a cell culture setting to determine its resistance to inhibition by a selected anti-Tetherin and its ability to inhibit viral release. In this way, chimeric proteins with optimized properties can be identified prior to use in vivo.

In addition to containing a recombinant nucleic acid and/or chimeric protein, cells of the invention often also contain one or more viruses, viral nucleic acids, and/or viral proteins. Typically, the viruses, viral nucleic acids, and/or viral proteins include those for which the recombinant nucleic acids and chimeric proteins are designed to counteract. For example, cells containing a chimeric protein that specifically inhibits release of HIV can also contain HIV viruses, nucleic acids, and proteins, including anti-Tetherin proteins.

The recombinant nucleic acids, chimeric proteins, and cells of the invention have many uses. One aspect of the invention is directed to use of the nucleic acids, chimeric proteins, and/or cells in treatment of viral infections. As discussed above, certain cells produce Tetherin proteins, which inhibit release of enveloped viruses, such as HIV and KSHV, by binding to and retaining budding viruses at the cell surface. These viruses have evolved anti-Tetherin molecules to counteract the Tetherin proteins. The present invention is directed at reducing or eliminating the anti-Tetherin activity in virally infected cells by providing a Tetherin-derived chimeric protein that is active against viral release but resistant to inhibition by anti-Tetherins produced by the virus. In a broad sense, the method of treating viral infections includes providing a chimeric construct according to the invention to a virally infected cell, which results in reduction or elimination of viral release from the cell. In embodiments, the chimeric protein is supplied to the cell exogenously. In other embodiments, the chimeric protein is supplied to the cell endogenously by way of expression of a recombinant nucleic acid.

The method of treating can be understood from various points. In one view, the method is a method of treating a cell infected with a virus. In another view, the method is a method of treating a subject infected with a virus. In yet another view, the method is a method of eliminating or reducing the amount of a virus in a cell. In yet a further view, the method is a method of eliminating or reducing the amount of virus in a subject infected with the virus. According to each view of the method, a chimeric protein is provided to a virally infected cell to reduce or eliminate release of the virus from the cell. Where the method is practiced in vitro or ex vivo, the step of providing the chimeric protein can be by way of direct addition of the protein to a cell culture and allowing the protein to insert into the cell membrane of infected cells. Alternatively, the step of providing can be by way of insertion of a recombinant nucleic acid into the cell and expression of a chimeric protein from the recombinant nucleic acid. When practiced in vivo, the step of providing can be by way of administering to a subject a chimeric protein or recombinant nucleic acid, where the administration can be systemic (e.g., by injection or transfusion) or can be local (e.g., by direct injection into infected tissue).

In embodiments, the method is an in vitro method of treatment of one or more cells that are infected with a virus. The method of treatment includes exposing at least one infected cell to a chimeric protein under conditions that allow the protein to associate with and insert into the cellular membrane. Insertion into the cellular membrane blocks release of virions from the cell and effects transient treatment of the cell. The act of exposing can be any act that results in the chimeric protein contacting the infected cell. It thus may be addition of the protein to cell culture media in which the infected cell is found. Alternatively, it may be by way of associating the chimeric protein with one or more substances that facilitate contact with the cellular membrane and/or insertion into the cellular membrane. For example, a chimeric protein can be provided as part of a liposome or other lipid-containing complex, or can be provided in a complex with an antibody that can target the complex to a particular cell surface molecule.

In alternative embodiments of the in vitro method, the chimeric protein is delivered to the infected cell by way of delivery of a recombinant nucleic acid to the infected cell. Once taken up by the infected cell, the recombinant nucleic acid expresses a chimeric protein, which is inserted into the cell membrane and effects treatment by reducing or eliminating viral release from the cell. Delivery of the nucleic acid can be by any suitable technique, including, but not limited to transfection of nucleic acid into the cell using electroporation, chemical delivery, or delivery by way of viral infection and insertion of the recombinant nucleic acid as part of a viral genome. Insertion of the recombinant nucleic acid into the cell can cause transient expression of the chimeric protein, for example through extrachromosomal expression of the coding region for the chimeric protein. Alternatively, expression of the chimeric protein can be stable and long-term by way of integration of the coding region for the chimeric protein into the genome of the infected cell. Various techniques for transient and permanent expression of heterologous nucleic acids are known in the art, and the practitioner may select any suitable technique.

In embodiments, the method is an in vivo method of treatment. As mentioned above, in vivo treatment can be by way of administering a chimeric protein to a subject. Embodiments relating to in vivo treatment with the chimeric protein utilize well-known and widely practiced techniques for delivery of biologics. Those of skill in the medical arts are aware of such techniques, and can practice such techniques without undue or excessive experimentation.

Treatment in vivo can also be accomplished by administration of a recombinant nucleic acid of the invention to a subject suffering from a viral infection. In essence, these in vivo treatment methods can be considered methods of gene therapy that provide a therapeutic treatment for viral infections. In general, the methods of gene therapy include administering a recombinant nucleic acid of the invention to a subject suffering from a viral infection, which results in uptake of the recombinant nucleic acid into at least the infected cells, and expression of a chimeric protein of the invention. Expression of the chimeric protein reduces or eliminates viral release, and effects treatment of the subject for the viral infection. Delivery and uptake into the cell preferably results in stable integration of the recombinant nucleic acid into infected cells; however, the invention encompasses transient expression, for example by way of extrachromosomal elements having the coding sequence for the recombinant protein. Various techniques for in vivo gene therapy are known in the art. The practitioner may select any suitable technique for use in the present invention.

In particularly preferred embodiments, the method of treating is a gene therapy method that is practiced ex vivo. More specifically, gene therapy techniques have shown great promise when cells to be treated are removed from the subject's body, treated in vitro, and returned to the subject's body. Such methods are referred to herein as ex vivo treatments. Treatment methods performed ex vivo combine the power of nucleic acid insertion into target cells in vitro with the long-term expression of integrated recombinant sequences in vivo. Furthermore, insertion of recombinant nucleic acids in vitro can eliminate the need for the use of viral vectors and the problems associated with them. Additionally, in vitro insertion of recombinant nucleic acids allows for assay for successful integration of the recombinant sequences prior to reintroduction of cells into the subject.

Ex vivo therapeutic methods include: removing target cells from the body of the subject to be treated; introducing recombinant nucleic acid molecules into the cells; and returning the treated cells to the subject's body. In embodiments, the methods can include one or more of the following actions: purifying target cells from one or more other cells present in the sample taken from the subject's body, either prior to or after treatment; screening for cells that have incorporated the recombinant nucleic acid; and enriching the treated cell population for cells that express the chimeric protein.

One advantage to ex vivo methods as compared to purely in vivo methods is the ability to select the target cell population. Whereas in purely in vivo methods, recombinant nucleic acids are delivered systemically or locally to a tissue that includes target cells, the ex vivo methods of the invention allow for improved selection of target cells. The ex vivo methods thus reduce introduction of recombinant nucleic acids into non-target cells and reduce the associated side-effects that might accompany expression in non-target cells.

In an exemplary embodiment of ex vivo gene therapy treatment, HIV infection in a subject is provided. In this exemplary embodiment, bone marrow cells are extracted from a subject and a recombinant nucleic acid of the invention stably inserted into the cells. The cells are returned to the subject's body, where they recolonize the bone marrow. Differentiation of the cells into blood cells results in populating the subject's body with recombinant blood cells. Recombinant T cells are thus present in the subject, and the subject is rendered resistant to HIV infection. The method can be practiced using the steps outlined above or can be practiced with additional steps. For example, after removal of bone marrow cells for treatment, the subject may be further treated to ablate white blood cells from the body, thus reducing HIV load and the subject's reservoir of HIV infected cells. As such, repopulation with HIV-resistant blood cells will result in reduction or elimination of the virus from the subject. Such a method, while advantageously practiced on bone marrow cells, can also be practiced on differentiated blood cells, such as a population of mixed white blood cells, a population of mixed T cells, or a specific subset of T cells.

Those of skill in the art will immediately recognize the advantages provided by the invention as they relate to ex vivo gene therapy treatments for numerous viral diseases. The concepts broadly described herein with regard to chimeric proteins and recombinant nucleic acids can be applied to any number of enveloped viruses that rely on anti-Tetherin activities. Likewise, the specific examples provided herein with regard to HIV can be applied to other viruses and target cell populations to effect treatment for any number of viral infections.

### EXAMPLES

The invention will be further explained by the following Examples, which are intended to be purely exemplary of the invention, and should not be considered as limiting the invention in any way.

### Example 1

### Production and Use of Chimeric Tetherin Proteins

Tetherin is a protein that restricts the release of enveloped viruses from cells by tethering the viruses as the cell surface. The human Tetherin protein has been shown to be active against a variety of enveloped viruses, including retroviruses, Ebola, HIV, and arenaviruses. HIV-1 counteracts Tetherin through the action of its Vpu protein. This Example provides chimeric proteins (referred to herein as “TT” and “MT”) that are resistant to Vpu and therefore represent anti-HIV-1 biologicals. The TT and MT constructs are also resistant to the KSHV K5 protein. These particular molecules represent a prototype of a new class of anti-viral compounds based on virus-resistant Tetherin derivatives.

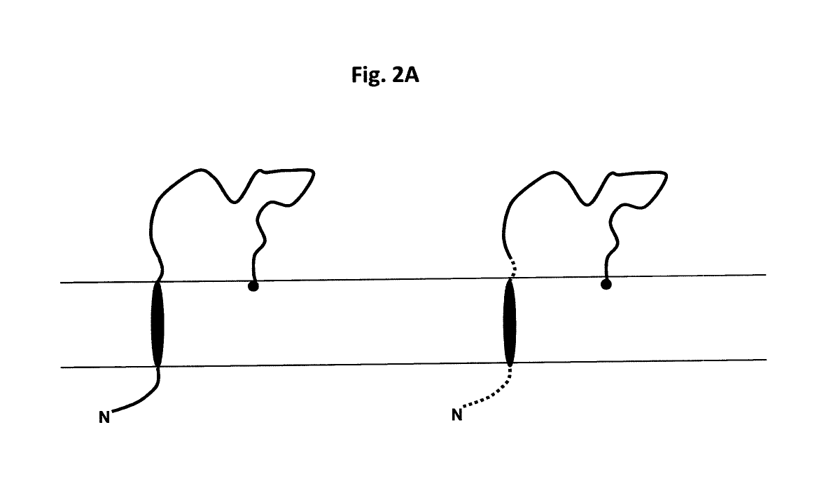

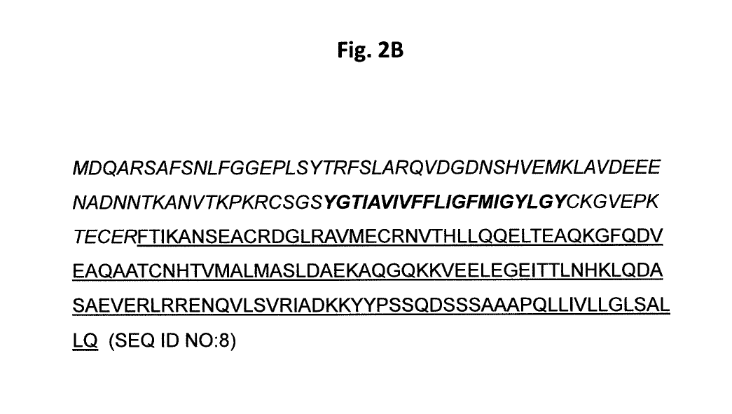

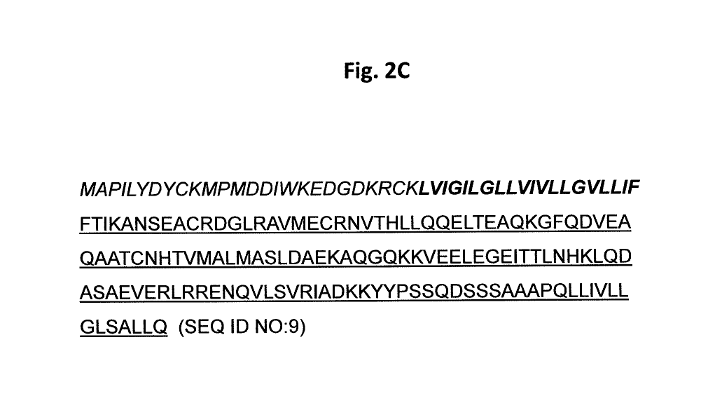

A chimeric protein was constructed using the extracellular domain of human Tetherin and a portion of human Transferrin Receptor type I protein (TfR1). More specifically, residues 44-180 of the human Tetherin protein were fused at their N-terminus to the cytoplasmic tail and transmembrane domain of TfR1. The construct was designated as “TT” and is depicted in cartoon fashion in FIG. 2A, and the primary amino acid sequence provided in FIG. 2B. A similar chimeric protein was created using the human Tetherin ectodomain and the cytoplasmic tail and transmembrane domain of macaque tetherin. The construct was designated “MT”, and its primary amino acid sequence is provided in FIG. 2C and as SEQ ID NO:9.

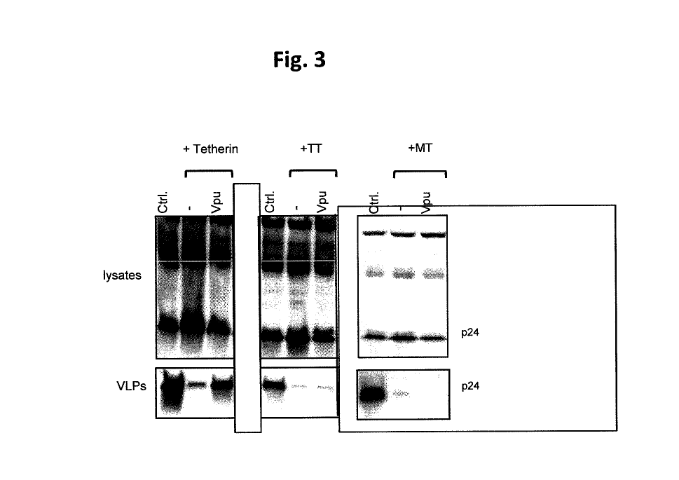

The chimeric proteins were tested for their ability to block release of Virus Like Particles (VLP) from infected cells. The results are depicted in FIG. 3. More specifically, cells of human cell line 293 were transfected with HIV-1 Gag-Pol-Rev expression plasmids that generate virus-like particles that are released into the supernatant. In a parallel procedure, the cells were co-transfected with human Tetherin expression plasmids. In another parallel procedure, the cells were co-transfected with both a “TT” expression plasmid and an HIV Vpu expression plasmid. In yet another parallel procedure, the cells were co-transfected with both an “MT” expression plasmid and an HIV Vpu expression plasmid. The VLPs from each cell transfection procedure were concentrated from the respective supernatants by ultracentrifugation. Western blotting of cell lysates and VLP pellets using anti-HIV-1-p24 antibodies was then performed. Such a technique provides an indication of the extent of HIV-1 particle release from the cells by assaying p24 release.

The left panel of FIG. 3 shows that expression of the Gag-Pol-Rev plasmid resulted in significant VLP release from the cells (heavy p24 band). Co-expression of human Tetherin essentially eliminates VLP release. However, expression of Vpu restores VLP release. In summary, the addition of Tetherin decreases VLP release but this is counteracted by Vpu.

The center panel shows that the TT construct functions as a Tetherin with regard to virus release, but is not counteracted by Vpu. More specifically, expression of the Gag-Pol-Rev plasmid results in significant VLP release from the cells (heavy p24 band). Co-expression of both Tetherin and TT (independently) restrict VLP release. However, unlike Tetherin, TT restriction of VLP release is not relieved by Vpu. As such, the TT chimeric protein functions as a Tetherin for virus release, but is resistant to Vpu inhibition.

The right panel shows that, like the TT construct, the MT construct functions as a Tetherin with regard to virus release, but is not counteracted by Vpu. More specifically, expression of the Gag-Pol-Rev plasmid results in significant VLP release from the cells (heavy p24 band). Co-expression of both Tetherin and MT (independently) restrict VLP release. However, unlike Tetherin, MT restriction of VLP release is not relieved by Vpu. As such, the MT chimeric protein functions as a Tetherin for virus release, but is resistant to Vpu inhibition

This set of experiments shows that the TMC of Tetherin is involved in regulation of its activity by the anti-Tetherin HIV-1 Vpu. Other data (not shown) provides similar support with regard to the KSHV K5 anti-Tetherin protein. Substitution of the Tetherin TMC significantly reduced or abolished the inhibitory effect of anti-Tetherins on the protein. Yet, at the same time, the anti-viral activity of the Tetherin portion was retained via the extracellular domain. It is thus shown that the TMC, is sufficient and necessary for inhibition of activity of Tetherins by anti-Tetherins. Chimeric proteins having active Tetherin extracellular domains but lacking wild-type Tetherin TM or TMC can thus be produced as anti-viral compounds, which can be expressed endogenously in virally infected cells.

It is recognized herein that the particular Tetherin sequences for each species of organism have specificity for particular anti-Tetherins from viruses that specifically infect those species. The comparison given in FIG. 1 guides those of skill in the art in selecting which residues to alter, if desired, for a given species, to reduce/abolish anti-Tetherin activity or to maintain anti-Tetherin activity. More specifically, a requirement for specific sequences in the Tetherin membrane spanning domain is shown by the fact that replacing the TM or TMC region with the equivalent region from the human Transferrin receptor protein (TfR) leads to a Tetherin derivative that retains the ability to block virus release, but is no longer counteracted by the HIV-1 Vpu anti-Tetherin protein. In addition, replacing the membrane spanning domain of human Tetherin with the equivalent region from the rhesus macaque Tetherin produces a Tetherin protein that retains the ability to block virus release, but is no longer counteracted by the HIV-1 Vpu anti-Tetherin protein, despite the substantial homology between these two sequences. Similarly, replacing the cytoplasmic tail of Tethein with the region from TfR blocks the ability of KSHV K5 to counteract the protein (data not shown). Certain specific sequences in Tetherin are therefore required for the interaction with anti-Tetherin proteins, and the present disclosure provides those of skill in the art with the guidance needed to select mutations that achieve a desired goal.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1. A chimeric protein comprising:
a first amino acid sequence sufficient for localizing the protein to a surface of a cell; and
a second amino acid sequence that interacts with and tethers an enveloped virus to the surface of the cell,
wherein the cell-surface localizing activity of the first amino acid sequence is not substantially inhibited by an anti-Tetherin protein expressed by the virus,

2. The chimeric protein of claim 1, wherein the N-terminus of the first amino acid sequence is fused to the C-terminus of the second amino acid sequence, directly or by way of a linker peptide.

3. The chimeric protein of claim 2, further comprising a third sequence fused at its C-terminus to the N-terminus of the second sequence.

4. The chimeric protein of claim 3, wherein the first sequence is a human transmembrane domain sequence and the third sequence is a human cytoplasmic tail sequence derived from the same protein as the first sequence.

5. The chimeric protein of claim 3, wherein the first sequence comprises a transmembrane domain sequence from a human Transferrin Receptor Type I protein and the third sequence comprises a cytoplasmic tail sequence from the human Transferrin Receptor Type I protein.

6. The chimeric protein of claim 3, wherein:
the first sequence comprises a transmembrane domain sequence from a human Transferrin Receptor Type I protein;
the second sequence comprises a human Tetherin protein extracytoplasmic domain; and
the third sequence comprises a cytoplasmic tail sequence from the human Transferrin Receptor Type I protein.

7. The chimeric protein of claim 1, wherein the second sequence comprises a human Tetherin protein extracytoplasmic domain.

8. The chimeric protein of claim 1, wherein the chimeric protein comprises the sequence of SEQ ID NO:8.

9. A recombinant nucleic acid encoding a chimeric protein comprising:
a first amino acid sequence sufficient for localizing the protein to a surface of a cell; and
a second amino acid sequence that interacts with and tethers an enveloped virus to the surface of the cell,
wherein the cell-surface localizing activity of the first amino acid sequence is not substantially inhibited by an anti-Tetherin protein expressed by the virus.

10. The recombinant nucleic acid of claim 9, which is part of a construct for expression of the chimeric protein.

11. The recombinant nucleic acid of claim 10, wherein expression of the chimeric protein is under the control of a cell-specific promoter or the HIV-1 LTR promoter.

12. An in vitro cell, said cell comprising a chimeric protein comprising:
a first amino acid sequence sufficient for localizing the protein to a surface of a cell; and
a second amino acid sequence that interacts with and tethers an enveloped virus to the surface of the cell,
wherein the cell-surface localizing activity of the first amino acid sequence is not substantially inhibited by an anti-Tetherin protein expressed by the virus.

13. (canceled)

14. (canceled)

15. The cell of claim 12, further comprising the enveloped virus, a viral nucleic acid of the enveloped virus, or a viral protein of the enveloped virus.

16. An ex vivo method of gene therapy, said method comprising:
removing target cells from a subject;
inserting a recombinant nucleic acid into at least one target cell, wherein the recombinant nucleic acid encodes a chimeric protein comprising a first amino acid sequence sufficient for localizing the protein to a surface of a cell; and a second amino acid sequence that interacts with and tethers an enveloped virus to the surface of the cell, wherein the cell-surface localizing activity of the first amino acid sequence is not substantially inhibited by an anti-Tetherin protein expressed by the virus; and
reintroducing the treated cells into the subject,
wherein at least some of the reintroduced cells express the chimeric protein, thus providing an anti-viral effect in the subject.

17. The method of claim 16, wherein inserting the recombinant nucleic acid into at least one target cell comprises stably inserting at least the coding region for the chimeric protein into the target cell genome.

18. The method of claim 16, wherein the target cells are bone marrow cells or white blood cells.

19. The method of claim 16, wherein the target cells are bone marrow cells, the enveloped virus is HIV-1, and the second sequence of the chimeric protein comprises a human Tetherin extracellular domain sequence.

20. The method of claim 16, wherein the target cells are bone marrow cells, the enveloped virus is HIV-1, and the chimeric protein comprises the sequence of SEQ ID NO:8.

21. The chimeric protein of claim 1, wherein the anti-Tetherin protein is the HIV-1 Vpu protein, the HIV-2 Env protein, or the KSHV K5 protein.

22. The recombinant nucleic acid of claim 9, wherein the anti-Tetherin protein is the HIV-1 Vpu protein, the HIV-2 Env protein, or the KSHV K5 protein.


SEQUENCE LISTING (9 sequences)

SEQ ID NO: 1 - protein, 180 aa, Homo sapiens
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 2 - protein, 180 aa, Chlorocebus aethiops; identical to SEQ ID NO: 1

SEQ ID NO: 3 - protein, 182 aa, Macaque fasicularis
MAPILYDYCKMPMDDIWKEDGDKRCKPVIGILGLLVIVLLGVLLIFFTIKANSEACQDGLRAVMECRNVTYLLQQELAEAQRGFRDAEAQAVTCNQTVMALMASLDAEKAQGRKKVEELEGEITTLNHKLQDASAEVERLRRENHVLNARIADTDSASSQDSSCAAEPPLLILLLGLSALLL

SEQ ID NO: 4 - protein, 181 aa, Macaque mulatta
MAPILYDYCKMPMDDIWKEDGDKRCKLVIGILGLLVIVLLGVLLIFFTIKANSEACQDGLRAVMECRNVTYLLQQELAEAQRGFRDAEAQAVTCNQTVMALMASLDAEKAQGRKKVEELEGEITTLNHKLQDSAEVERLRRENHVLNARIADTDSGSSQDSSCAAEPPVLILLLGLSALLL

SEQ ID NO: 5 - protein, 181 aa, Macaque nemistrina
MAPILYDYCKMPMDDIWKEDGDKRCKLVVGILGLLVIVLLGVLLIFFTIKANSEACQDGLRAVMECRNVTYLLQQELAEAQRGFRDAEAQAVTCNQTVMALMASLDAEKAQGRKKVEELEGEITTLNDKLQDSAEVERLRRENHVLNARIADTDSASSQDSSCAAEPPLLILLLGLSALLL

SEQ ID NO: 6 - protein, 184 aa, Pan troglodytes
MASTLYDYCRVPMDDIWKKDGDKRCKLLLGIGILMLLIIVILGVPLIIFTIKANSEACRDGLRAVMECRNVTHLLQQELTEAQKGFQDVEAQAATCNHTVMALMASLDAEKAQGQKKVEELEEEITTLNHKLQDSAEVERLRRENQVLSVRIADKKYYSSSQDSSSAAAPQLLIVLLGLSALLQ

SEQ ID NO: 7 - DNA, 1479 bp, Artificial Sequence
  Fusion coding sequence comprising human transferrin receptor type I C-terminus and TM domain fused to human Tetherin ectodomain
atggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtccggaggtcatggtactggttctactggttctggttcttctagatctcgagctgatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagattcaccatcaaggccaacagcgaggcctgccgggacggccttcgggcagtgatggagtgtcgcaatgtcacccatctcctgcaacaagagctgaccgaggcccagaagggctttcaggatgtggaggcccaggccgccacctgcaaccacactgtgatggccctaatggcttccctggatgcagagaaggcccaaggacaaaagaaagtggaggagcttgagggagagatcactacattaaaccataagcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtga

SEQ ID NO: 8 - protein, 234 aa, Artificial Sequence
  Chimeric protein containing human Transferrin ectodomain fused to human transferrin receptor type I transmembrane domain and cytoplasmic tail
MDQARSAFSNLFGGEPLSYTRFSLARQVDGDNSHVEMKLAVDEEENADNNTKANVTKPKRCSGSYGTIAVIVFFLIGFMIGYLGYCKGVEPKTECERFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 9 - protein, 183 aa, Artificial Sequence
  Chimeric protein containing macaque Tetherin cytoplasmic tail and transmembrane regions fused to human Tetherin extracellular region
MAPILYDYCKMPMDDIWKEDGDKRCKLVIGILGLLVIVLLGVLLIFFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ